(19) United States
(12) Patent Application Publication
(10) Pub. No.: US 2024/0026945 A1
(43) Pub. Date: Jan. 25, 2024

(54) HUMAN ADAPTABLE VARIABLE STIFFNESS SPRINGS

(71) Applicant: VANDERBILT UNIVERSITY, Nashville, TN (US)

(72) Inventors: David Braun, Nashville, TN (US); Chase W. Mathews, Nashville, TN (US)

(21) Appl. No.: 18/226,204

(22) Filed: Jul. 25, 2023

Related U.S. Application Data
(60) Provisional application No. 63/391,854, filed on Jul. 25, 2022.
(60) Provisional application No. 63/405,229, filed on Sep. 9, 2022.

(51) Int. Cl. F16F 1/14; F16F 1/16

(52) U.S. Cl. CPC F16F 1/145; F16F 1/16

(57) ABSTRACT

Various examples of systems, methods, and applications of variable stiffness springs are described. In one example, a variable stiffness joint apparatus can include a torsional spring; a variable stiffness mechanism comprising a self-locking mechanism and a linkage system, the self-locking mechanism comprising an auxiliary spring; and an actuator in communication with the auxiliary spring of the self-locking mechanism. When the actuator changes position, a force is applied to the auxiliary spring by the actuator and a stiffness is adjusted at an energy cost that is independent of the stiffness of the spring and the energy stored by the spring. In another example, a self-adjusting variable stiffness mechanism can include a compression spring. The energy stored by compressing the compression spring and the mechanism can self-adjust a stiffness to enable energy accumulation using a same maximal compression force which is not dependent on the energy accumulated in the spring.

### CROSS-REFERENCE TO RELATED APPLICATIONS

This application claims the benefit of and priority to U.S. Provisional Application No. 63/391,854, titled “HUMAN ADAPTABLE VARIABLE STIFFNESS SPRINGS,” filed on Jul. 25, 2022, and U.S. Provisional Application No. 63/405,229, titled “METHOD AND APPARATUS FOR ITERATIVE ENERGY ACCUMULATION USING SPRINGS SUBJECT TO FORCE AND DEFORMATION CONSTRAINTS,” filed on Sep. 9, 2022, the entire contents both of which applications are hereby incorporated herein by reference.

### BACKGROUND

A conventional spring has constant stiffness that defines how much force it exerts upon deflection and how much energy it stores upon deflection. The stiffness of a spring depends on the material, shape, and size of the spring. Variable stiffness springs change their shape or size to increase or decrease stiffness; provide more or less force and store more or less energy upon the same deflection.

### BRIEF DESCRIPTION OF THE DRAWINGS

Many aspects of the present disclosure can be better understood with reference to the following drawings. The components in the drawings are not necessarily drawn to scale, with emphasis instead being placed upon clearly illustrating the principles of the disclosure. In the drawings, reference numerals designate corresponding parts throughout the several views.

FIG. 1A illustrates an isometric view of an example human-selectable variable stiffness spring apparatus according to various embodiments described herein.

FIG. 1B illustrates a section view of the example human-selectable variable stiffness spring apparatus shown in FIG. 2A according to various embodiments described herein.

FIG. 2A illustrates a model of the human-selectable variable stiffness spring apparatus of FIG. 1A according to various embodiments described herein.

FIG. 2B illustrates a schematic representation of the variable stiffness spring of FIG. 2A according to various embodiments described herein.

FIG. 3 illustrates an example of the human-selectable variable stiffness spring apparatus including a control device according to various embodiments described herein.

FIG. 4 illustrates an example of torque-angle characteristics of the variable stiffness spring of FIG. 1A according to various embodiments described herein.

FIG. 5A illustrates an example of the self-locking pivot point mechanism according to various embodiments described herein.

FIG. 5B illustrates the working principle of the self-locking pivot point mechanism in the configuration for increasing stiffness (left) and decreasing stiffness (right) according to various embodiments described herein.

FIG. 6A illustrates an example of an experimental set up according to various embodiments described herein.

FIG. 6B illustrates examples of the self-locking pivot point mechanism of FIG. 5A at various displacements according to various embodiments described herein.

FIG. 7 illustrates an example model of the variable stiffness floating spring-leg according to various embodiments described herein.

FIGS. 8A-8C illustrates an example of a floating variable stiffness spring implemented for the user to iteratively increase the energy stored by the spring according to various embodiments described herein.

FIG. 9A illustrates an example diagram of a mass-spring system of a floating variable stiffness spring according to various embodiments described herein.

FIG. 9B illustrates an example of force-deflection of the human leg in the example of FIG. 9A according to various embodiments described herein.

FIG. 10A illustrates an example of simulated behavior of force-deflection achieved by repeated squats for the spring-leg of FIG. 7 according to various embodiments described herein.

FIG. 10B illustrates an example of simulated behavior of potential energy stored by the spring of the spring-leg of FIG. 7 according to various embodiments described herein.

FIG. 11 illustrates front and top views an example of a floating spring-leg mechanism according to various embodiments described herein.

FIG. 12A illustrates an example model of a full assembly of the floating spring-leg mechanism according to various embodiments described herein.

FIGS. 12B and 12C illustrate an example model of spring compression according to various embodiments described herein.

FIGS. 12D and 12E illustrate an example model of change mechanical advantage according to various embodiments described herein.

FIG. 13 illustrates an example of the spring-leg mechanism at various stages according to various embodiments described herein.

FIGS. 14A and 14B illustrate experimental results of the spring-leg mechanism of FIG. 13 according to various embodiments described herein.

### DETAILED DESCRIPTION

In the context described above, various examples of systems, methods, and applications of a human adaptable variable stiffness springs are disclosed herein. In a non-limiting example, a variable stiffness spring apparatus can have a control device to allow a user to select a stiffness modulation without using precisely timed forces. For example, a variable stiffness joint apparatus is described where the stiffness of the joint can be changed by the human similar to manually changing gears in bicycles. In another non-limiting example, a variable stiffness spring apparatus can ensure that energy is accumulated in the spring after repeated compression of the spring. For example, a self-adjusting variable stiffness apparatus is described where the stiffness of the apparatus is changed automatically similar to the automatic gear shifting in cars.

Described below are various embodiments of the present systems and methods for human adaptable variable stiffness springs therefor. Although particular embodiments are described, those embodiments are only exemplary implementations of the system and method. One skilled in the art will recognize other embodiments are possible. All such embodiments are intended to fall within the scope of this disclosure. Moreover, all references cited herein are intended to be and are hereby incorporated by reference into this disclosure as if fully set forth herein. While the disclosure will now be described in reference to the above drawings, there is no intent to limit it to the embodiment or embodiments disclosed herein. On the contrary, the intent is to cover all alternatives, modifications, and equivalents included within the spirit and scope of the disclosure.

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth to provide those of ordinary skill in the art with a complete disclosure and description of how to construct and use the systems and methods disclosed and claimed herein. Efforts have been made to ensure accuracy with respect to numbers (e.g., amounts, etc.), but some errors and deviations should be accounted for.

It is to be understood that, unless otherwise indicated, the present disclosure is not limited to particular materials, manufacturing processes, or the like, as such can vary. It is also to be understood that the terminology used herein is to describe particular embodiments only and is not intended to be limiting. It is also possible in the present disclosure that steps can be executed in different sequences where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

In the following discussion, a general description of the systems of the present disclosure and their components is provided, followed by a discussion of the operation of the same. Non-limiting examples of human adaptable variable stiffness springs are discussed.

A conventional spring has constant stiffness that defines how much force it exerts upon deflection and how much energy it stores upon deflection. The stiffness of a spring depends on the material, shape, and size of the spring. Variable stiffness springs change their shape or size to increase or decrease stiffness, provide more or less force, and store more or less energy upon the same deflection.

Variable stiffness springs can be used as transmission mechanisms similar to the variable gear transmission in bicycles and cars. The benefit of variable stiffness spring transmission compared to variable gear transmission is that the former enables power amplification (or de-amplification), while the latter cannot provide power amplification (or de-amplification).

To demonstrate the power amplification capability of a variable stiffness spring, it is assumed that the spring is initially compressed by a motor, human limb, or any other force, such that it stores E Joules of energy. The average power of the spring is defined as, p=E/T, the ratio between the energy stored by the spring and the time T required by the spring to fully extend and release the stored energy. The time required by the spring to release the stored energy depends on the stiffness of the spring k because a spring with lower stiffness will need more time to fully extend compared to a spring with higher stiffness: k₁<k₂ implies T₁>T₂ (assuming that both springs are attached to the same mass). This example shows why and how variable stiffness springs can amplify power: k₁<k₂ implies p₁<p₂.

Power amplification (and de-amplification) is a beneficial feature of a transmission mechanism because power amplification (and de-amplification) can be used to achieve rapid acceleration (and breaking). For example, power amplification (and de-amplification) can be used to increase the productivity of an industrial robot, by speeding up (or slowing down) the robot performing a pick-and-place task. Also, power amplification can be used to speed up (or slow down) a robot exoskeleton assisting or augmenting human limbs in jumping, walking, running, swimming, or other everyday activities.

However, increasing the stiffness of a variable stiffness spring can cost a large amount of energy. For example, the minimum amount of energy needed to increase the energy stored by a linear helical spring k₁<k₂ is the elastic potential energy added to the spring while changing the spring stiffness:

$$Δ E = {E}_{2} - {E}_{1} = \frac{1}{2} ( {k}_{2} - {k}_{1} ) Δ {L}^{2} = \frac{{k}_{2} - {k}_{1}}{{k}_{1}} {E}_{1} .$$

While this formula suggests that increasing spring stiffness upon the same deflection will cost energy proportional to the energy stored by the spring ΔE˜E₁, it also shows that the stiffness of the spring could be changed at a low energy cost when the spring stores no energy E₁=0.

The reasoning above has led to prior designs of variable stiffness springs that afford low energy cost stiffness variation when the spring stores no energy.

Disclosed herein is a simple control method that leverages this feature in the context of human assistance during walking with a variable stiffness hip joint. The method has the benefit of only requiring a small control force to change the stiffness of a variable stiffness spring because it changes stiffness when the spring stores no energy E₁=0 (when the variable stiffness joint is undeflected). However, in many applications, condition E₁=0 can only be met at isolated time points during oscillatory motion, and therefore, the method has a practical limitation of requiring precisely timed control forces when the spring stores no energy or when it stores a limited amount of energy E₁≈0.

The requirement for precisely timed control forces imposes a limitation on the practical realization of low energy cost stiffness modulation in variable stiffness springs. For example, it imposes a limitation on the practical realization of a variable stiffness spring with human changeable stiffness.

Variable stiffness springs with human changeable stiffness are springs where the stiffness change is done by an effortless finger, hand, arm, or limb movement. Variable stiffness springs with human changeable stiffness are similar to the variable gear transmission mechanism used in bicycles and cars, as the latter afford gear shifting with not only small force but also a slow finger or limb motion.

To address the limitation of using precisely timed forces for low energy cost stiffness modulation, a novel variable stiffness spring is disclosed. The key element of the design is the series spring directly connected to the variable mechanical advantage mechanism that changes stiffness. The series spring affords a small force to vary the stiffness of the spring to be generated at any time during the motion, for example, the time when the spring stores energy during oscillatory motion. In this way, the series spring removes the requirement of a precisely timed force to enable low energy cost stiffness variation.

An example embodiment and a use case of the series elastic spring-driven variable stiffness spring is described herein. The series spring can be used to convert previously designed variable stiffness springs into a variable stiffness spring that affords stiffness variation using small and imprecisely timed forces. The series spring is not limited to devices where that stiffness is varied by a human; it can be used in variable stiffness springs where the stiffness is varied by a motor. In the latter case, the series spring changes the requirement of a weak and fast motor to a weak and slow motor; a motor that has a lower peak power requirement.

The benefit of a variable stiffness spring transmission compared to variable gear transmission is that the former enables power amplification (or de-amplification), while the latter cannot provide power amplification (or de-amplification). The human adaptable variable stiffness springs requirement for precisely timed control forces imposes a limitation on the practical realization of low energy cost stiffness modulation in variable stiffness springs. For example, it imposes a limitation on the practical realization of a variable stiffness spring with human changeable stiffness. Variable stiffness springs with human changeable stiffness are springs where the stiffness is changed by an effortless finger, hand, arm, or limb movement. Variable stiffness springs with human changeable stiffness are similar to the variable gear transmission mechanism used in bicycles and cars, as the latter afford gear shifting with not only small force but also a slow finger or limb motion.

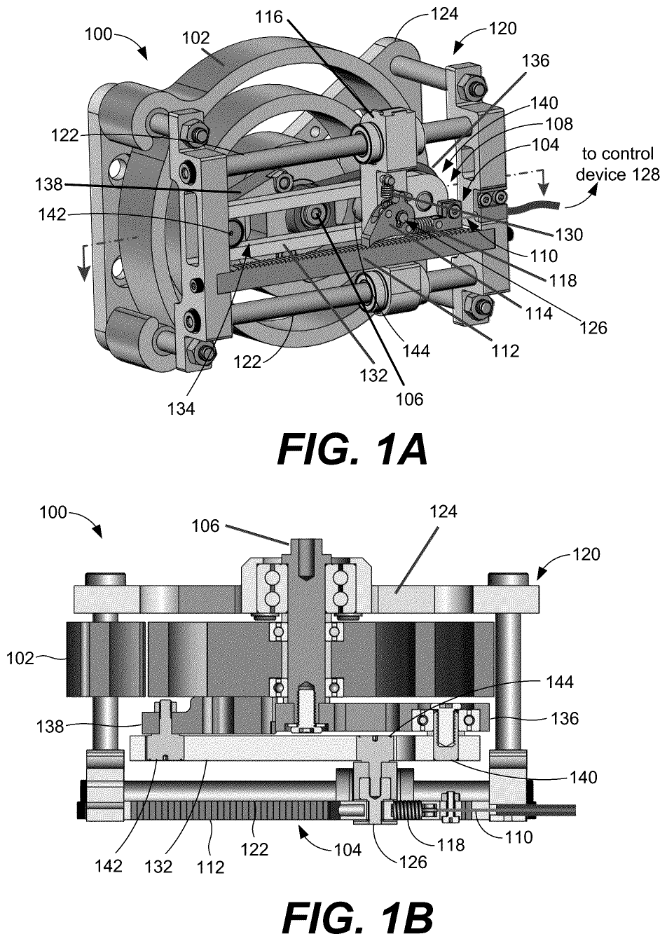

Turning to FIGS. 1A and 1B, shown are an isometric view of an example human-selectable variable stiffness spring apparatus 100 (FIG. 1A) and a section view (FIG. 1B). The human-selectable variable stiffness spring apparatus 100 can include a torsional variable stiffness spring 102 and a self-locking pivot point mechanism 104. For example, the torsional variable stiffness spring 102 can be a spiral torsion spring (also referred to as a torsional spring, torsional joint spring, or joint spring herein). The torsional variable stiffness spring 102 can be mounted about a shaft 106. The human-selectable variable stiffness spring apparatus 100 can also include a linkage system 108 arranged to contact the torsional spring 102 and change a stiffness of the torsional spring 102. The human-selectable variable stiffness spring apparatus 100 also includes an actuator 110. The self-locking pivot point mechanism 104 is suitable to be incrementally controlled by the actuator 110.

The torsional variable stiffness spring 102 can include a spiral torsion spring 102. In an example, the torsional variable stiffness spring 102 can be a 3D printed carbon fiber reinforced spiral torsion spring 102 due to customizability and large volumetric energy density. The spiral torsion spring 102 can be fabricated using Onyx (nylon filled with chopped carbon fiber) with half of the layers reinforced with continuous strands of carbon fiber. The self-locking pivot-point mechanism 104 and linkage system 108 can be used to vary the mechanical advantage of the spiral spring over the joint.

The self-locking pivot point mechanism 104 can include a ratchet-pawl mechanism that includes a linear ratchet 112 and a pawl 114. The self-locking pivot point mechanism 104 can also include a pawl support 116 and an auxiliary spring 118 (also referred to as a series spring herein). The linear ratchet 112 can be arranged orthogonal to and offset from the shaft 106. The linear ratchet 112 and pawl support 116 can be coupled to a housing 120, where the linear ratchet 112 is stationary and the pawl support 116 can translate along housing supports 122 that are arranged parallel to the linear ratchet 112. The housing 120 can also include a mounting plate 124 from which the shaft 106 extends and a means to connect the housing supports 122 to the mounting plate 124. The pawl 114 coupled to the pawl support 116 at a pivot point 126 and configured to engage with the linear ratchet 112. The pawl 114 can engage with the linear ratchet 112 and pivot about the pivot point 126 to translate along the linear ratchet 112.

The self-locking pivot point mechanism 104 also includes a pawl-release spring 130 (also referred to as a parallel spring herein). The pawl-release spring disengages the pawl from the ratchet. The pawl-release spring 130 is attached to the pawl 114 at an offset to the pivot point 126 and connected to the pawl support 116. The auxiliary spring 118 and pawl-release spring 130 are positioned at an offset from a pivot point 126 of the pawl 114 such that the auxiliary spring 118 and pawl-release spring 130 generate opposing moments on the pawl 114. Together, the auxiliary spring 118 and pawl-release spring 130 can control the engagement of the pawl 114 with the linear ratchet 112 to enable the user to change the stiffness of the torsional spring 102.

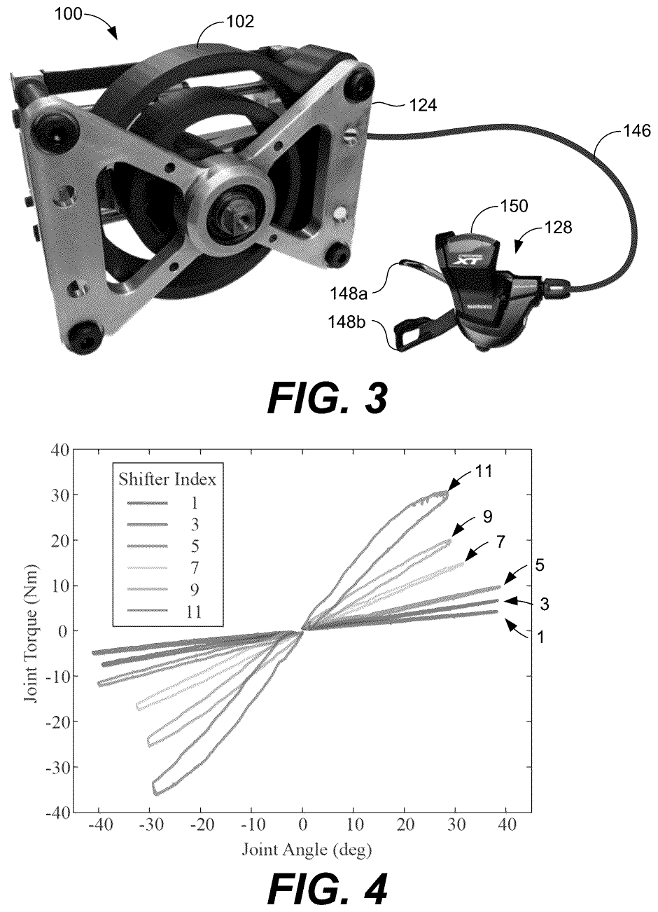

The auxiliary spring 118 is coupled in series between the actuator 110 and the pawl 114. A user can control the actuator 110 via a connected control device 128 suitable for manual control of the actuator 110. The user can select to increase or decrease the stiffness of the torsional variable stiffness spring 102 without precise timing. A level of stiffness of the torsional variable stiffness spring 102 based at least in part on a position of the pawl 114 with respect to the linear ratchet 112. When the actuator 110 changes position, a force is applied to the auxiliary spring 118 by the actuator. For example, the actuator 110 can comprise a Bowden cable 146 extending from a manual control device 128 (FIG. 3), which will be described in further detail. When a user extends or retracts the actuator (or cable) 110, a force and a moment are generated via the auxiliary spring 118 to pivot the pawl 114 about the pivot point 126 to engage or disengage the pawl 114 from the linear ratchet 112. When an applied force of the actuator 110, and the force generated by the auxiliary spring 118 is larger than the force of the pawl-release spring 130 and the reaction force of the joint spring 102, the pawl 114 is moved to increase the stiffness of the torsional spring 102. When an applied force of the actuator 110, and the force generated by the auxiliary spring 118 is smaller than the force of the pawl-release spring 130 and the reaction force of the joint spring 102, the pawl 114 is moved to decrease the stiffness of the torsional spring 102. While a cable is shown as the actuator 110 in an example, other types of actuators can be relied on to displace the auxiliary spring 118.

The linkage system 108 can be arranged between the torsional variable stiffness spring 102 and the self-locking pivot point mechanism 104 to modulate the stiffness of the torsional spring 102. The linkage system 108 can include a lever arm 132 having a slot 134, a first linkage arm 136, and a second linkage arm 138. The first linkage arm 136 is rotatable about shaft 106 and pivotably coupled by a first pin 140 to a first end portion of the lever arm 132. The second linkage arm 138 is rotatable about shaft 106 and can contact the torsional spring 102. The distal end of the second linkage arm 138 can have a second pin 142 at a distal portion of the second linkage arm 138 that is arranged to slide within slot 134 of the lever arm 132. The pawl support 116 can have a support pin 144 that extends from a surface opposite along the same axis of the pivot point 126 which supports the pawl 114. The lever arm 132 can be arranged such that the support pin 144 of the pawl support 116 of the self-locking pivot point mechanism 104 is positioned within the slot 134 of the lever arm 132. The lever arm 132 is positioned to pivot about the same axis as the pivot point 126 of the self-locking pivot point mechanism 104 and to vary the mechanical advantage of the spiral spring 102.

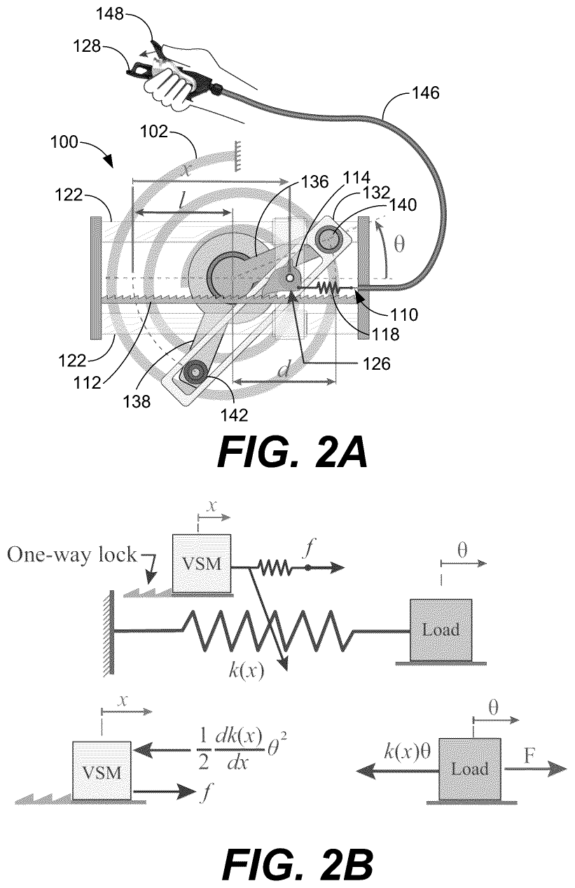

As shown in FIGS. 2A and 3, the variable stiffness joint apparatus 100 can include a control device 128 suitable for manual control of the actuator 110 by a user. For example, the actuator 110 of the control device can include a Bowden cable 146. The control device 128 can include at least one lever 148 to modulate stiffness by moving the actuator 110. The control device 128 can be handheld and operable by a finger of the user, requiring only a small force by the user. The user can incrementally select the stiffness via the at least one lever 148. In some examples, the control device 128 can have a first lever 148a to increment stiffness and a second lever 148b to decrement stiffness. In some examples, the control device 128 can have an index indicator 150 to display a value to indicate a level of stiffness. Although a Bowden cable 146 and handheld control device 128 are shown as an example, other configurations and implementations of user-controlled actuators can be relied on.

The method of changing the stiffness of a torsional variable stiffness spring 102 can include applying a force to the auxiliary spring 118 of a self-locking pivot point mechanism 104. The force can be applied by the actuator 110 to provide a positive or negative force on the auxiliary spring 118. Applying a force to an auxiliary spring 118 of a self-locking pivot point mechanism 104 can include changing a position of the actuator 110 that acts on the auxiliary spring. In an example, the actuator 110 can be controlled by a user applying small forces via a handheld control device 128. For example, when a user extends or retracts the actuator 110, a force and a moment are generated via the auxiliary spring 118 to pivot the pawl 114 about the pivot point 126 to disengage the pawl 114 from the linear ratchet 112. When an applied force of the actuator 110, and the corresponding moment generated by the auxiliary spring 118 about the pivot point 126 is larger than the moment generated by the pawl release spring 130, and the force generated by the auxiliary spring 118 is larger than the reaction force applied by the joint spring 102 to the support pin 144, the pawl 114 translates along the linear ratchet 112 to increase the stiffness of the torsional spring 102. When an applied force of the actuator 110, and the corresponding moment generated by the auxiliary spring 118 about the pivot point 126 is smaller than the moment generated by the pawl release spring 130, and the force generated by the auxiliary spring 118 is smaller than the reaction force applied by the joint spring 102 to the support pin 144, the pawl 114 translates along the linear ratchet 112 to decrease the stiffness of the torsional spring 102.

The self-locking pivot point mechanism 104 is coupled to the torsional variable stiffness spring 102 via the linkage system 108. Changing a position of the pivot point 126 of the self-locking pivot point mechanism 104 is based at least in part on the force applied to the auxiliary spring 118 which can cause the stiffness of the variable stiffness spring to change.

Examples of the design of human-selectable variable stiffness spring apparatuses 100 are discussed in further detail below. For example, variable stiffness springs with human-selectable stiffness can be used as a human-adjustable variable stiffness joint. The stiffness is adjusted at an energy cost that is independent of the stiffness of the spring and the energy stored in the spring. The stiffness can be adjusted without large or precisely timed forces provided by a motor or human.

Springs are commonly used in wearable robotic devices to provide assistive joint torque without the need for motors and batteries. However, different tasks (such as walking or running) and different users (such as athletes with strong legs or the elderly with weak legs) necessitate different assistive joint torques, and therefore, springs with different stiffness. Variable stiffness springs are a special class of springs which can exert more or less torque upon the same deflection, provided that the user is able to change the stiffness of the spring. Presented herein is a novel variable stiffness spring design in which the user can select a preferred spring stiffness similar to switching gears on a bicycle. A leg-swing experiment demonstrates that the user can increment and decrement spring stiffness in a large range to effectively assist the hip joint during leg oscillations. Variable stiffness springs with human-selectable stiffness could be key components of wearable devices which augment locomotion tasks, such as walking, running, and swimming.

Mechanical springs are commonly used in wearable devices to provide assistive torque without the use of motors and batteries. Prior works have shown that unpowered spring-driven exoskeletons can reduce the metabolic cost of walking by 7% and running by 8%; in both cases, a fixed stiffness spring was optimized across users. Alternatively, a variable stiffness spring with selectable stiffness could enable users to choose the most optimal spring stiffness for the task, similar to how a bicycle derailleur enables cyclists to select the most optimal gear ratio independent of the cyclist and the cycling speed.

Variable stiffness springs have been previously used in lower limb prostheses and orthoses to help humans with motions such as walking, running, and stair-ascent. Many of the previously designed variable stiffness springs ensure that a small force can be used to adjust the spring stiffness when the spring is unloaded. However, for oscillatory motions such as the swing of the hip during walking, running, or swimming, the spring may only be at equilibrium for a fraction of a second during each cycle of the motion. Consequently, if the human aims to effortlessly change stiffness during an oscillatory task, then the human must precisely apply the force to change stiffness when the spring is not deflected at each cycle.

Using a variable stiffness robot actuator, a small but fast motor can change spring stiffness during oscillatory motion, once each motion cycle, by applying precisely timed forces. Replacing the motor with a human limb and a bicycle hand shifter is impractical because the user would be required to operate the hand shifter at precise times. However, by placing a spring in series between the hand shifter and the mechanism which adjusts spring stiffness, the requirement for precisely timed movements can be eliminated. The variable stiffness spring with human-selectable stiffness replaces a weak but fast motor with a human finger to control the stiffness.

The variable stiffness mechanism described herein enables the human to change the stiffness of the spring in the same way the derailleur enables cyclists to change the gear ratio of the bicycle. The device consists of a 3D-printed composite spiral spring and a variable stiffness mechanism. The stiffness of the spring is changed by a series-spring actuated Bowden cable hand shifter, and a unique self-locking mechanism implemented with a linear ratchet and pawl. The device allows the user to effectively change the assistance provided to the user in a leg swing experiment, where the hip joint of the human is augmented with the variable stiffness spring. The user can also increment and decrement the spring stiffness using a bicycle hand shifter, the same way a cyclist would down-shift or up-shift the gear ratio to adapt to different terrains and speeds while riding the bicycle.

Model—Human-Driven Variable Stiffness Spring Joint

The model of the human-driven variable stiffness spring joint is shown in FIG. 2A. For example, the variable stiffness spring with human-selectable stiffness 100 comprises the hand shifter 128, series spring 118, and the self-locking variable stiffness mechanism which includes a self-locking pivot point mechanism 104 and the linkage system 108. The joint is composed of a spring 102 and a mechanism that changes the stiffness of the spring. The free-body diagram of the model is a schematic representation of the variable stiffness spring shown in FIG. 2B.

The torque-angle relation of the variable stiffness spring, as shown in FIG. 2B, is defined by:

τ=τ(x, θ)≈k(x)θ, (1.1)

where τ is the joint torque, θ is the joint angle, k is the joint stiffness, while x denotes the position of the pivot point 126.

The joint stiffness k(x) depends on the design of the mechanism. The stiffness of the torsional spring 102, shown in FIG. 2A, is given by the following relation:

$$k ( x ) = \frac{\partial τ}{\partial θ} {\vert}_{θ = 0} \approx {{k}_{s} ( \frac{x}{l + d - x} )}^{2} , \qquad ( 1.2 )$$

where kₛ is the constant stiffness of the torsional spring attached to the joint, d is the length of the first linkage arm 136 attached to the shaft, while l is the length of the second linkage arm 138 attached to the spring 102. According to (1.2), the stiffness of the joint is a monotonically increasing function of the position of the pivot point 126, x.

Changing the position of the pivot point 126 changes the mechanical advantage of the spring 102 over the joint. The equation governing the motion of the pivot point is given by:

$$m \ddot{x} = f - F ( θ , x ) = f = \frac{1}{2} \frac{dk ( x )}{dx} {θ}^{2} , \qquad ( 1.3 )$$

where m is the mass of the stiffness modulating mechanism, f is the external force applied to modulate joint stiffness, while F is the reaction force of the spring aiming to move the pivot point 126 by back-driving the stiffness modulating mechanism (see FIG. 2B). The latter effect appears because the spring always tends to move the pivot-point to the position associated with the lowest joint stiffness.

In order to prevent the reaction force of the spring 102 from changing the position of the pivot point 126, a spring-loaded linear ratchet-pawl mechanism 112, 114 (FIG. 2A) is used. The mechanism locks when the force required to move the pivot point f exceeds the threshold locking force f₀ of the spring-loaded pawl; it is unlocked otherwise.

When the ratchet-pawl mechanism 112, 114 is locked, the reaction force of the joint spring 102 cannot move the pivot point 126 in the direction that lowers the joint stiffness,

0<f₀<f⇒\tilde{x}≥0, (1.4)

but the externally applied force f can be used to move the pivot point and increase the joint stiffness, given that the externally applied force is larger than the reaction force of the spring, f>F(θ, x) in (1.3).

When the ratchet-pawl mechanism 112,114 is unlocked 0<f<f₀, the reaction force of the spring can be used to lower the joint stiffness under the following condition 0<f<f₀<F(θ, x) in (1.3). This condition will be satisfied when the spring is considerably deflected, as in that case, the reaction force of the spring is much larger than the threshold force f₀ used to lock the ratchet-pawl mechanism 112,114.

Working Principle

The physical requirements to change the joint stiffness using small forces are examined below.

A variable stiffness joint placed at the human hip and attached to the leg is considered. It is further assumed that the leg swings back and forth with frequency ω, and amplitude θₘₐₓ, in walking or running,

θ=θₘₐₓ sin ωt. (1.5)

To increase the joint stiffness, an external force f that is larger than the spring force opposing the motion of the pivot point is applied,

$$f > F ( θ , x ) = \frac{1}{2} \frac{dk ( x )}{dx} {θ}^{2} ⇒ \ddot{x} > 0. \qquad ( 1.6 )$$

This condition can be satisfied by an arbitrarily small force f, when the spring is un-deflected, θ=0, or a small force when the spring is slightly deflected, θ≈0.

It is assumed that the force provided by the human is limited,

0≤f≤fₘₐₓ, (1.7)

and using (1.5) and (1.6), the time available to change the stiffness of the joint using the maximal force that can be provided by the human can be estimated by

$$\frac{Δ t}{T} < \frac{2}{π} \sqrt{\frac{{f}_{max}}{{F}_{max}}} , \qquad ( 1.8 )$$

where T=2π/ω is the period of leg oscillations.

Relation (1.8) suggests that if the maximal force to change the spring stiffness fₘₐₓ is small compared to the maximal reaction force of the spring Fₘₐₓ, then the time window in which the joint stiffness can be changed Δt is also small:

$$\frac{{f}_{max}}{{F}_{max}} ≪ 1 ⇒ \frac{Δ t}{T} ≪ 1. \qquad ( 1.9 )$$

Therefore, the timing of the external force f must be precise in order to increase the spring stiffness with a small force.

The requirement for precise timing is circumvented in our device by using a spring between the hand shifter and the pivot point, see FIG. 2A (series spring).

In a typical work cycle, the human would actuate the hand shifter once or multiple times in order to reduce the length of the Bowden cable, and consequently extend the series spring of stiffness kₛ, until the maximum force is reached,

f=kₛΔx≤fₘₐₓ. (1.10)

If the ratchet-pawl mechanism 112,114 is unlocked, then extending the spring will move the pivot point and increase the joint stiffness. If the ratchet-pawl mechanism 112,114 is locked, extending the spring will increase the force in the series spring but will not move the pivot point or increase the joint stiffness. Since the series spring can be extended while the ratchet-pawl mechanism is locked (1.6), the human can use the hand shifter over nearly the whole period of oscillations T, except perhaps the short time window Δt when the ratchet-pawl mechanism 112, 114 unlocks and the series spring 118 moves the pivot point 126 to increase the joint stiffness.

$$\frac{T - Δ t}{T} \approx 1. \qquad ( 1.11 )$$

Consequently, the time available for the human to apply force is not limited to Δt, and is largely independent of the force applied by the human.

In summary, the series spring removes the precise timing requirement to change the joint stiffness using small forces. The series spring also enables the user to extend the spring over multiple oscillation cycles, which further mitigates the precise timing required to change the joint stiffness without the series spring (1.9), or the time available to change stiffness during a single cycle with the series spring (1.11).

In the example shown in FIGS. 1A and 1B, the torsional variable stiffness spring 102 can be a 3D printed carbon fiber reinforced spiral torsion spring 102 due to customizability and large volumetric energy density. The spiral torsion spring 102 can be fabricated using Onyx (nylon filled with chopped carbon fiber) with half of the layers reinforced with continuous strands of carbon fiber. For example, the carbon fiber spring can have an estimated stiffness of kₛ≈24 Nm/rad. The adjustable pivot-point linkage mechanism 104 can be used to vary the mechanical advantage of the spiral spring over the joint.

In FIG. 4, experimental results of measured torque-angle data is shown. A static deflection experiment was performed to estimate the torque-deflection behavior of the variable stiffness joint. During the experiment, the spring was deflected at six different stiffness configurations.

The joint is characterized by the following torsional stiffness values,

k∈[6,70] nm/rad. (1.12)

This stiffness enables the spring to provide ±[3, 36] Nm assistive joint torque at ±30 deg joint rotation, which amounts to around 35% of the average human hip torque for a 75 kg person during walking at normal speeds (1.6 m/s).

One example of a control device 128 comprises a hand shifter (FIG. 3). The hand shifter 128 and the locking mechanism 104 can be used to change the stiffness and hold the torque provided by the torsional spring 102. The hand shifter has two levers that, when pressed, either extend or retract the Bowden cable. In an example, the combined mass of the hand shifter 128 and Bowden cable 146 is about 0.3 kg. The Bowden cable is a steel cable routed inside a flexible housing. In the bicycle, the Bowden cable shifts the chain between different sets of sprockets.

Bowden cables have been extensively used in wearable devices to transfer force. In our device, the two ends of the Bowden cable are fastened to the hand shifter and the series spring, while the series spring is connected to the pawl which can rotate about the pivot point, as shown in FIG. 1A. By changing the length of the Bowden cable, the human generates a force and a moment about the pivot point. The moment generated about the pivot point disengages the pawl from the ratchet, such that the force can move the pivot point. In this way, using the hand shifter and the Bowden cable, the human can change the stiffness of the joint.

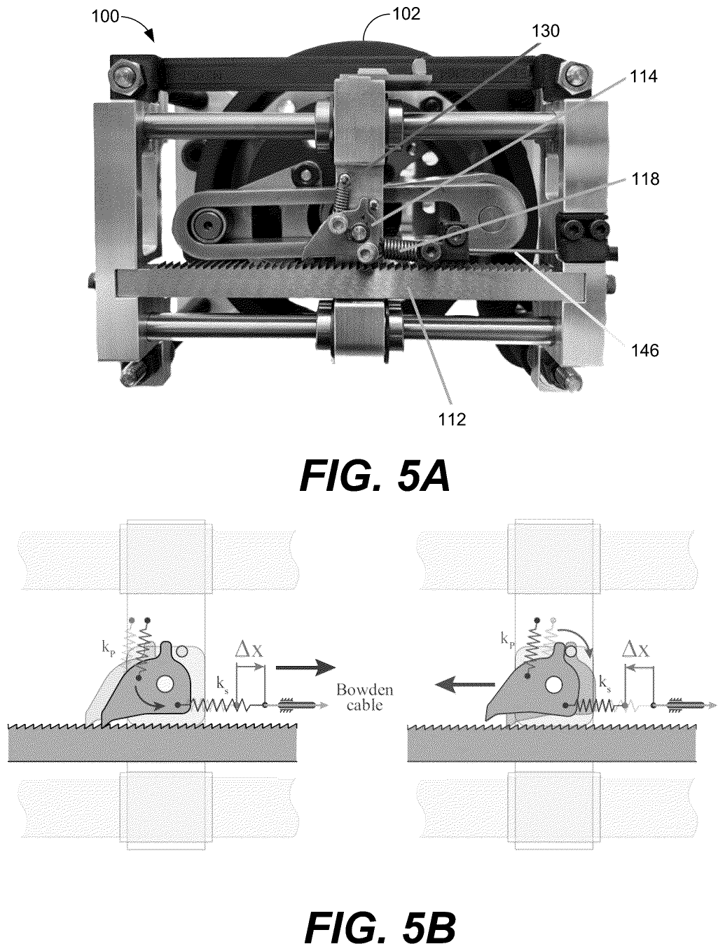

In the human-selectable variable stiffness spring apparatus 100, the pivot point self-locks through the use of a linear ratchet-pawl mechanism 112, 114, shown in FIGS. 5A and 5B. The mechanism which changes stiffness includes a linear ratchet rack 112, a pawl 114, a series spring 118 between the pawl 114 and the Bowden cable 146, and a parallel spring 130 between the pawl 114 and the pivot-point housing 120. The pawl 114 engages with the ratchet 112 to prevent the motion of the pivot point 126 when the large spiral spring 102 is loaded. The series spring 118 and parallel spring 130 of stiffness kₛ and kₚ (see FIG. 5B) control the engagement of the pawl 114 to enable the user to change the stiffness of the joint.

To increase stiffness, the user generates a force on the Bowden cable 146, which deflects the series spring 118. When the large spiral spring 102 is un-deflected, the series spring 118 pulls the pawl 114 and consequently translates the pivot point 126 (see FIG. 5B-right). The stiffness of the series spring kₛ≈6000 N/m is chosen small enough such that the user can extend it, but large enough such that it can move the pivot point when the large spiral spring is unloaded.

To decrease stiffness, the user extends the Bowden cable 146, which causes the cable to slack. When the cable slacks, the series spring 118 becomes unloaded, which allows the parallel spring 130 to disengage the pawl 114. In this case, the force imposed by the large deflected spiral spring 102 can move the pivot point, to reduce the joint stiffness, until the slack of the Bowden cable 146 is removed and the pawl 114 is re-engaged (FIG. 5B—left). The stiffness of the parallel spring 130 kₚ≈485 N/m has been chosen large enough to disengage the pawl 114 around zero joint deflection and small enough to allow the series spring 118 to re-engage the pawl 114 upon a small joint deflection.

### EXAMPLE 1

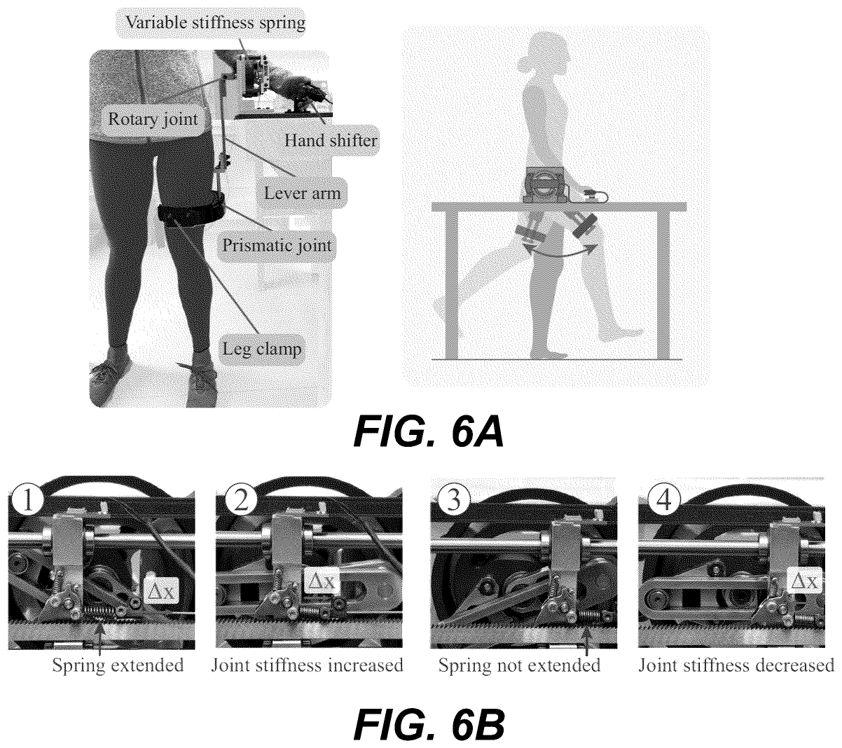

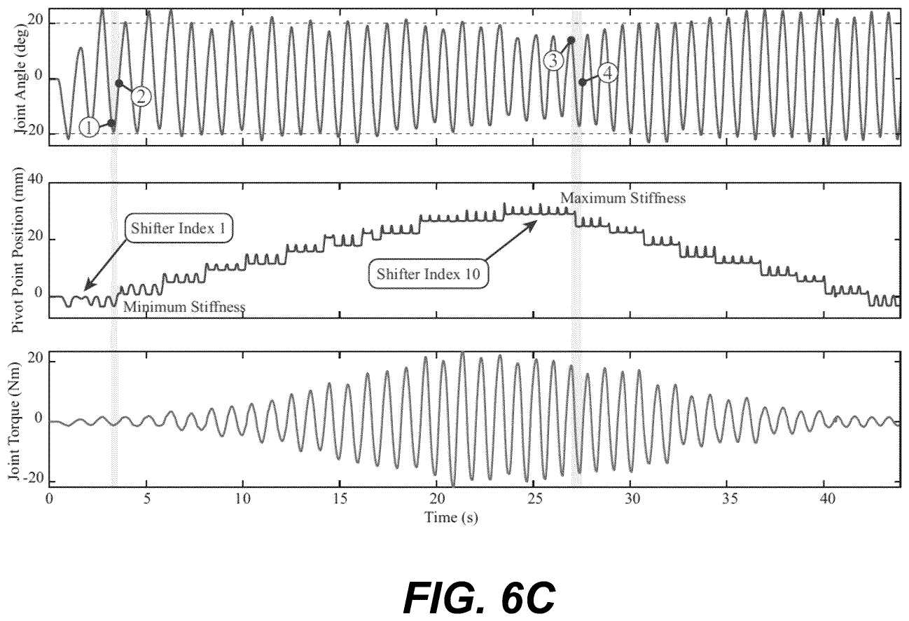

The human-selectable variable stiffness spring joint is used to augment the human hip joint (shown in FIG. 6A) in a leg swing experiment. The purpose of the experiment is to validate the working principle of the device which enables the human to quickly change joint stiffness over a large range during continuous oscillations.

To verify the function of the device, a desk-mounted setup is used, where the variable stiffness spring joint can be attached to the leg of a human, as shown in FIG. 6A. The leg of the subject was fastened to the spring using a 3D-printed clamp, while the clamp was connected to the joint using a lever arm. The lever arm features a rotary joint and a prismatic joint such that the leg is not constrained to move in the lateral and longitudinal directions while being attached to the joint.

The hip angle θ was captured using a rotary magnetic encoder. The position of the pivot point x was measured using a linear magnetic encoder. The torque provided by the variable stiffness joint was estimated using the measured joint angle θ, the measured pivot point position x, and the experimental torque-deflection curves shown in FIG. 4.

A simple exploratory experiment was performed, where the subject was asked to (i) swing one leg continuously back and forth at a comfortable frequency and a relatively constant amplitude of ±20 degrees, (ii) fully increment the bicycle shifter until the maximum stiffness has been reached, and subsequently, (iii) fully decrement the shifter until the minimum stiffness was reached. The protocol was approved by the Institutional Review Board of Vanderbilt University Medical Center (N220192).

The experimental data is shown in FIG. 6C. The mechanism is shown in FIG. 6B while the joint stiffness was incremented (top) and decremented (bottom). The angle of the hip joint is shown in FIG. 6C (top). The position of the pivot point is shown in FIG. 6C (middle). The joint torque is shown in FIG. 6C (bottom).

FIG. 6B shows snapshots of the device during the experiment with the corresponding timestamps labeled in FIG. 6C. The left snapshot shows an example of a stiffness increment where the series spring is first extended while the leg is away from equilibrium; in this case, the mechanism is locked, and subsequently, the series spring pulls the pivot point to a higher stiffness configuration when the leg is around equilibrium (FIG. 6B—top). The top snapshot shows an example of a stiffness decrement where the Bowden cable is slacked and the parallel spring lifts the pawl until the pivot point moves to a lower stiffness configuration and the slack of the Bowden cable is removed (FIG. 6B—bottom).

FIG. 6C (top) shows the joint angle during the experiment. The subject was able to generate continuous oscillatory leg motion such that the amplitude of the joint angle was around ±20 degrees.

FIG. 6C (middle) shows the position of the pivot point, and therefore the joint stiffness, during the experiment. The stiffness was increased from the minimum value (shifter index 1, where k(x)≈6 Nm/rad) to the maximum value (shifter index 10, where k(x)≈70 Nm/rad) during the oscillatory motion. The position of the pivot point oscillates approximately ±2 mm for each stiffness configuration. This oscillation was caused by the return spring which by default disengages the pawl in order to decrement stiffness as described herein. The oscillations did not cause detrimental effects during the experiment.

FIG. 6C (bottom) shows the estimated joint torque of the device during the experiment. As the user increased the joint stiffness of the spring, the torque of the spring was also increased from 1 Nm to around 20 Nm.

In summary, the experiment demonstrated that (i) the locking mechanism was able to successfully hold a given joint stiffness configuration as the device generated 1-20 Nm joint torque, and (ii) the user was able to effectively use the hand shifter to increment and decrement joint stiffness in a relatively large range of 6-70 Nm/rad, during continuous swings. The results show that the proposed device enables the human to adapt joint stiffness using an intuitive interface provided by the hand shifter of the bicycle.

A novel human-adjustable variable stiffness joint is described in which the user can select different joint stiffness, similar to cyclists selecting different gear ratios on a bicycle. The device enables the human to use small and non-precisely timed forces to change joint stiffness, rather than requiring the user to provide large forces or small but precisely timed forces. The ability of the human-adjustable variable stiffness joint to maintain and change stiffness during continuous oscillatory motion is also shown.

Customization of robot exoskeleton assistance for different users, tasks, and speeds has been shown to reduce metabolic demand in walking and running and may be useful for other everyday tasks. Individualized joint stiffness values could provide benefits to users across different speeds of walking. Individualized joint stiffness values may also help improve joint motion patterns and correct a reduced joint motion range of users with impairment. Finally, individualized joint stiffness values may be used to better augment users performing physically demanding tasks, such as lifting, jumping, running, or walking with a heavy load.

Human-adjustable variable stiffness joints can be key components of mechanically adaptive robot exoskeletons where different users can choose between different levels of assistance for different locomotion tasks.

### Energy Accumulation Under Force and Deformation Constraints

In another example, the above concepts can be extended and adapted to different scenarios. As a non-limiting example, a variable stiffness joint apparatus can be an uncontrolled mechanical device or mechanical automaton. The stiffness of a spring can be changed automatically with a kinematics design of a mechanism to ensure that energy is accumulated in the spring after repeated compression of the spring. As will be described in more detail, the self-adjusting variable stiffness mechanism can accumulate an increased amount of energy upon repeated compression with a constant maximal force, whereas a regular spring can only accumulate more energy by increasing the maximal compression force. Consequently, in the self-adjusting variable stiffness mechanism, each stroke can have the same maximal force, such that repeated spring compression does not require a larger force for each new stroke. In a non-limiting example, a device or apparatus using iterative energy accumulation as described herein can be a device wearable by a user, such as an exoskeleton, where repetitive actions allow for energy accumulation. However, the concepts can be relied upon for other devices that do not require direct human interaction. For example, the device or apparatus can be relied upon for other configurations and applications. In another non-limiting example, the concepts can be applied to tools or as part of a transmission mechanism attached to a motor to drive heavy machinery by first accumulating energy with a small torque-limited motor and then releasing the accumulated energy to generate significant force beyond what would be possible using the same motor.

Springs can provide force at zero net energy cost by recycling negative mechanical work to benefit motor-driven robots or spring-augmented humans. However, humans have limited force and range of motion, and motors have a limited ability to produce force. These limits constrain how much energy a conventional spring can store and, consequently, how much assistance a spring can provide. An approach to accumulating negative work in assistive springs over several motion cycles is discussed. By utilizing a novel floating spring mechanism, the weight of a human or robot can be used to iteratively increase spring compression, irrespective of the potential energy stored by the spring. Decoupling the force required to compress a spring from the energy stored by a spring could enable spring-driven robots and humans to perform physically demanding tasks without the use of large actuators.

Springs can enable robots actuated by motors and humans “actuated by muscles” to perform physically demanding tasks with reduced force requirements from the actuators. Typically, a spring is compressed slowly over a longer period of time, while the energy stored by the spring is released rapidly. In this way, the spring provides power amplification beyond what a motor-driven robot or “muscle-actuated” human can do without the assistance of a spring. However, the energy stored by a spring is limited by the maximum force used to compress the spring. Consequently, the maximal force that a robot or human can generate limits the amount of energy a spring can store, and the level of assistive benefit a spring can provide. This limitation may be alleviated by leveraging the energy storage ability of springs over multiple loading and unloading cycles instead of a single cycle.

In mechanical resonance, the benefit of springs is leveraged over multiple cycles of energy storage and release, instead of a single cycle. A familiar example is a pogo-stick, essentially a spring in series with the human legs, that allows the user to jump repeatedly to accumulate energy and reach jump heights much greater than in a single jump. To accomplish such a feat, the pogo-stick relies on iteratively increasing the kinetic energy of the human through multiple jumps. This increase in kinetic energy is required to generate large contact forces to compress the pogo-stick spring and thereby increase the energy stored by the spring. However, large forces are challenging for humans and robots to generate without increasing their kinetic energy.

A method and a device for iteratively accumulating energy using only the static gravitational force provided by the mass of a spring-driven robot or the mass of a human augmented with a spring leg exoskeleton are described. The method utilizes the repeated application of a constant static force that is independent of the energy stored by the spring. The method also relies on a new device, which belongs to the class of floating spring mechanisms. The device is an energetically passive variable stiffness spring which automatically adjusts its stiffness to ensure that a constant force can compress the spring regardless of how much energy is stored by the spring.

For example, a self-adjusting variable stiffness mechanism can include a compression spring, where energy is stored by compressing the compression spring and the mechanism self-adjusts a stiffness to enable energy accumulation using a same maximal compression force which is not dependent on the energy accumulated in the spring. Further, repeated compressions increase an amount of energy stored.

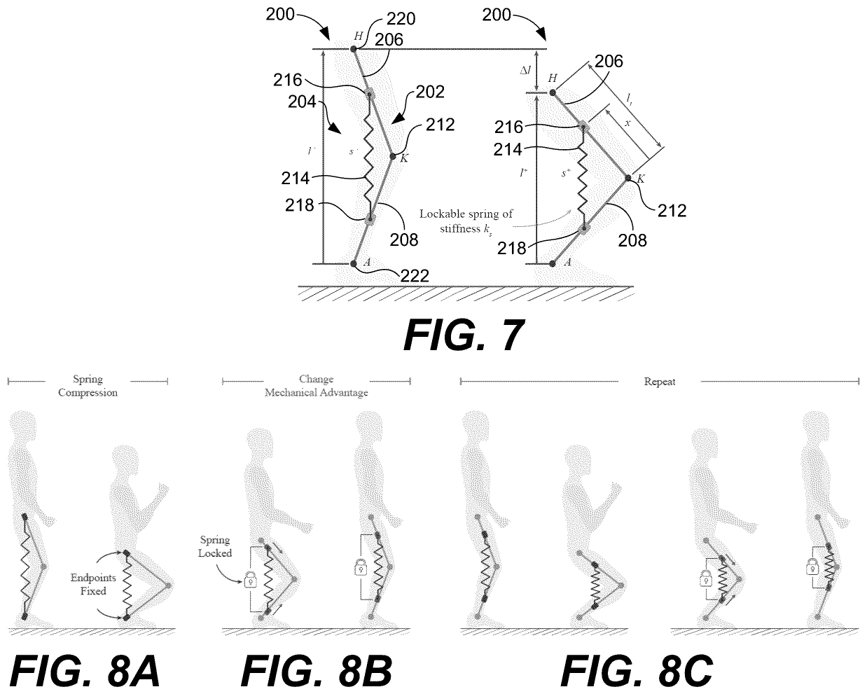

In FIG. 7, an example of a self-adjusting variable stiffness mechanism 200 is shown as a model of a lower-limb variable stiffness spring exoskeleton attached parallel to the legs of a user. The self-adjusting variable stiffness mechanism 200 can include a leg structure 202 and a floating spring assembly 204. The leg structure 202 can include first and second linear shafts 206, 208 coupled by a hinge joint 212. The floating spring assembly 204 can include a compression spring 214. The first and second ends 216, 218 of the floating spring assembly 204 can be slidably attached to the first and second linear shafts 206, 208, respectively. In this example, the first linear shaft 206 can have a free end 220 that can coincide with the hip (H) of a user, and the second linear shaft 208 can have a free end 222 that can coincide with the ankle (A) of a user. The first and second linear shafts 206, 208 can be configured such that the hinge joint 212 coincides with the knee (K) of the user. As shown in this example, the first and second linear shafts 206, 208, illustrated as leg segments H̅K̅ and K̅A̅, can be equal in length and the spring 214 is assumed to remain vertical (x is constant) as the leg deforms by Δl. While shown in this example as a model of a user-worn device, the concepts can be relied upon in other configurations and applications.

Energy Accumulation Using Springs

In an example, a simple energy accumulation task is considered, where the human is augmented with a spring exoskeleton attached parallel to the legs. In this task, the user compresses the spring by repeatedly squatting with the exoskeleton. The energy stored by the spring is retained by locking the spring at the bottom of each squat. As the human returns to the standing height, the spring shifts to a new configuration that grants the user a greater mechanical advantage over the spring for the next iteration. The greater mechanical advantage ensures the user can compress the spring at the beginning of each squat cycle until a desired amount of energy is accumulated in the spring. In the described iterative energy accumulation process, the force required by the human to achieve full spring compression is independent of the energy stored by the spring.

Shown in FIGS. 8A-8C is an example of repeated compression of a self-adjusting variable stiffness mechanism 200. As illustrated, an example squatting task using a simple spring-mass model of the human augmented with a conceptual lower-limb variable stiffness spring exoskeleton is shown. In this example, the relationship of the spring assembly 204 to the leg structure 202 is shown for select body positions of the user during repeated compressions. In FIG. 8A, in the first stage of spring compression the endpoints of the spring assembly 304 are fixed while the user compresses the spring 214 with a squat. In FIG. 8B, the endpoints of the spring are free while the spring is locked. The mechanical advantage of the user over the spring is increased as the user stands and the spring shifts towards the knee joint. In FIG. 8C, the user can iteratively increase the energy stored by the spring 214.

Basic Model—Single Squat

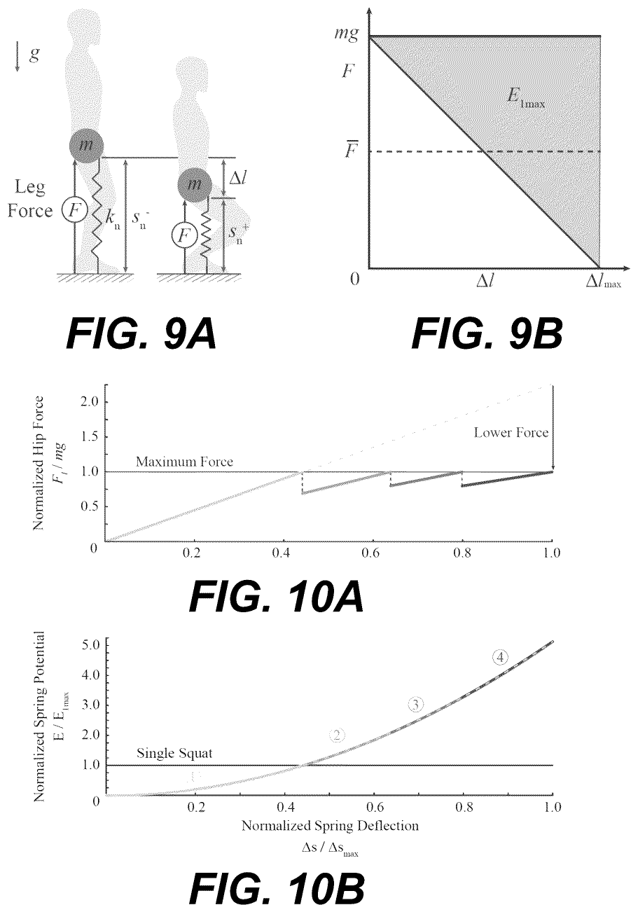

The human and the spring-leg exoskeleton are abstracted into a basic model shown in FIG. 9A. The human augmented with a lower limb exoskeleton is shown as a mass-spring system. The leg deformation is described as Δl. The body mass is supported by a spring with stiffness kₙ and deformed length sₙ^{±}; where the superscripts ± denote the pre-squat and post-squat spring lengths, respectively, and the subscript n denotes the number of squats performed during repeated squatting.

A single squat, starting from an upright standing position and ending at the fully squatted position is considered. Due to the geometric constraint of the leg, the leg deformation during a squat is given by

Δl ∈[0, Δlₘₐₓ]. (2.1)

Because the human leg can push but cannot pull against the ground while squatting, the force exerted by the leg on the center of mass must be positive,

F≥0. (2.2)

Finally, it is assumed that the human legs can produce enough force to stand up after each squat without the support of the exoskeleton. Any leg force that can overcome the weight of the human F≥mg suffices this assumption.

An example of the human leg force that enables squatting from a standing position to an equilibrium squat position is shown in FIG. 9B. The example force-deflection of the human leg is shown with the solid line that leads to an average leg force

$$\overline{F} = \frac{1}{2} mg$$

shown with the dashed line. The maximum amount of energy accumulated during one squat E_{1 max} is shown in the shaded area.

At standing the human limbs fully support the mass such that F=mg and the spring-leg exoskeleton does not provide any force. In the fully squatted position, the human limbs may support the mass with a force F ∈ [0, mg) while the spring leg provides the rest of the force required to keep the center of mass in static equilibrium,

mg=F+kΔlₘₐₓ. (2.3)

At the bottom of the squat, the energy stored by the spring leg depends on the human limb force. Assuming an average limb force F̅, the energy stored by the spring is given by:

$$\frac{1}{2} k Δ {l}_{max}^{2} = ( mg - \overline{F} ) Δ {l}_{max} . \qquad ( 2.4 )$$

In order to simultaneously satisfy (2.3) and (2.4), the average force of the human leg F̅ during the squat must satisfy the following condition:

$$\overline{F} = \frac{1}{2} ( mg + F ) . \qquad ( 2.5 )$$

According to (2.4) and (2.5), the maximum amount of energy that can be stored in the spring during a single squat is given by:

$${E}_{1 max} = \frac{1}{2} mg Δ {l}_{max} . \qquad ( 2.6 )$$

Relation (2.6) directly shows that the amount of energy accumulated in the spring is restricted by the range of motion and the limited gravitational force available to compress the spring.

Increasing the energy accumulated in the spring beyond the maximum amount of energy that can be stored in the spring during a single squat may be done by multiple squats. However, there are three main practical challenges to accumulating energy via multiple squats. First, the mechanism must provide increased mechanical advantage to compress the spring, such that the same force can be used to compress the spring even as the spring stores more energy. Second, the mechanism must provide controllable coupling between the spring and the leg, such that the same leg deformation can be used to input energy into the spring in subsequent squats independent of how much energy is stored by the spring. Third, to ensure efficient energy accumulation, the two prior tasks should be accomplished while maintaining the energy stored in the spring between subsequent iterations.

In the example shown in FIG. 7, the spring maintains a vertical orientation but shifts towards or away from the knee joint to change the mechanical advantage of the human over the spring. Consequently, the self-adjusting variable stiffness mechanismalters mechanical advantage by controlling the endpoints of the spring while the spring is locked, which maintains the potential energy stored by the spring. In turn, this ability to control the endpoints of the spring allows the leg deformation to be decoupled from the spring deformation, independent of the energy stored by the spring.

Model—Cyclic Energy Accumulation

FIG. 7 shows the floating spring variable stiffness leg for a single squat iteration. In the leg, points H, K, and A coincide with the user's hip, knee, and ankle, respectively. The thigh and shank segments HK and KA are assumed to be of equal length lt. The spring is also assumed to maintain its vertical orientation independent of the leg deformation. In the mechanism, the length of the spring s is defined by leg length l and the position of the spring x,

$$s = \frac{x}{{l}_{t}} l , \qquad ( 2.7 )$$

while the force required at the hip F₁ to compress the spring is defined by,

$${F}_{l} = ( \frac{x}{{l}_{t}} ) {F}_{S} = ( \frac{x}{{l}_{t}} ) {k}_{S} ( {s}_{0} - s ) , \qquad ( 2.8 )$$

where s₀ is the uncompressed length of the spring and kₛ is the stiffness of the spring.

These relations suggest that by moving the spring towards the knee joint—decreasing x—a small constant force F₁ could be used at the hip to compress the spring despite a potentially large spring force Fₛ, and consequently, the large amount of energy stored by the spring.

As a result, the mechanism shown in FIG. 7 could accumulate a large amount of energy when compressed by the weight of the human over multiple squats. In order to predict the behavior of the mechanism beyond a single squat, the simple example of a human performing a repetitive squat task to accumulate energy in the spring is considered as shown in FIGS. 8A-8C.

First, similar to (2.3), it is assumed that the spring-leg supports the weight of the user at the end of each squat,

$$mg = {k}_{s} ( \frac{{x}_{n}}{{l}_{t}} ) ( {s}_{0} - {s}_{n}^{+} ) . \qquad ( 2.9 )$$

To define the spring length at the end of the squat sₙ⁺, the spring location xₙ must be related to the spring length at the beginning of the squat sₙ⁻. The simple relation below follows from locking the spring length between the end of the previous squat and the beginning of the next squat (FIGS. 8A-8C),

sₙ₋₁⁺=sₙ⁻. (2.10)

According to (2.10), the energy stored by the spring will be retained between squats,

$$\frac{1}{2} {{k}_{s} ( {s}_{n - 1}^{+} - {s}_{0} )}^{2} = \frac{1}{2} {{k}_{s} ( {s}_{n}^{-} - {s}_{0} )}^{2} . \qquad ( 2.11 )$$

Finally, using (2.7) and (2.10), a recurrence relation that predicts the position of the spring across squat iterations is defined:

$${x}_{n} = \frac{{s}_{n}^{-}}{{s}_{n - 1}^{-}} {x}_{n - 1} . \qquad ( 2.12 )$$

Substituting (2.9), (2.10), and (2.12) into (2.8), it is found that the force required to compress the spring at the beginning of the next squat is always lower than the constant gravitational force available to compress the spring,

$${F}_{ln}^{-} = ( \frac{{s}_{n}^{-}}{{s}_{n - 1}^{-}} ) mg \leq mg . \qquad ( 2.13 )$$

FIG. 10A shows the force-deflection predicted during multiple squats, during which the maximal force provided by the human is bounded by the weight of the user. This force is compared to the force required to achieve the same spring deformation in a single squat (dashed line). Further, the vertical dashed lines show that the spring deformation is maintained between iterations, as required by (2.10).

FIG. 10B shows the energy accumulation process for the iterative method (gray), with energetic potential normalized by the maximum energy that can be achieved in a single squat provided by the weight of the user, E_{1 max}, as defined in (2.6). The four squats shown the minimum number of repeated squats to achieve the maximum spring deformation shown in FIG. 10B.

The spring accumulates the same amount of energy through four squats (gray) than in a single squat (gray dashed) but with less than half of the required force (FIGS. 10A and 10B). Furthermore, the reduction in force translates to over five times more stored energy as compared to what can be accumulated in a single squat subject to the same maximum force (FIG. 10B). Following the repeated squats, the spring can be reset to the initial mechanical advantage, x=lₜ, where the accumulated energy can be released to provide double the assistive force as compared to the maximal force used to compress the spring (FIG. 10A) and supply significantly more energy than what is stored by the spring when compressed with the maximal force in a single squat (FIG. 10B). The new functionality could allow a user to accumulate energy for lifting a large load, or enable a spring-driven robot to accumulate energy for increasing jump height.

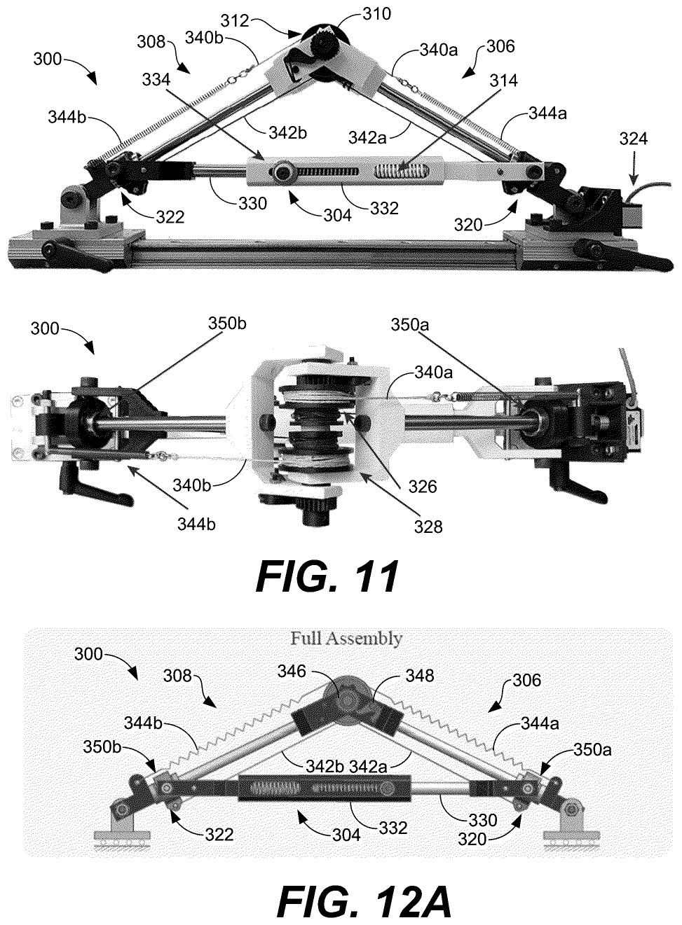

Turning to FIG. 11, an example implementation of a self-adjusting variable stiffness mechanism 300 is shown. The self-adjusting variable stiffness mechanism 300 includes the same features as the self-adjusting variable stiffness mechanism 200 and is described in further detail. Although the self-adjusting variable stiffness mechanism 300 is shown in an experimental setup, the features and concepts can be relied upon for other configurations and implementations, such as the exoskeleton discussed in relation to FIGS. 7-9.

As shown in FIG. 11, the self-adjusting variable stiffness mechanism 300 is shown in an experimental set-up. The self-adjusting variable stiffness mechanism 300 can include a leg structure 302 and a floating spring assembly 304. The leg structure 302 can include first and second linear shafts 306, 308 coupled by a joint pin 310 to form a hinge joint 312. The floating spring assembly 304 can include a compression spring 314. The first and second ends 316, 318 of the floating spring assembly 304 can be slidably attached to the first and second linear shafts 306, 308, respectively. In this example, the first linear shaft 306 can have a free end 320 connected to a load cell 324. For example, similar to the hip in FIG. 7, the free end 320 of the floating spring assembly 304 can be compressed and the force measured by the load cell 324. The second linear shaft 208 can have a free end 322 that is shown as fixed in this example. The self-adjusting variable stiffness mechanism 300 also includes first and second unidirectional pulleys 326, 328 mounted in coincidence with the joint pin 310. The first and second unidirectional pulleys 326, 328 can be configured to move the ends 316, 318 of the floating spring assembly 304 in unison.

The compression spring 314 in the floating spring assembly 304 can be compressed repeatedly to store energy. The floating spring assembly 304 can also include a piston 330, a cylinder 332, and a lock 334. The energy stored by the compression spring 314 is retained by locking the floating spring assembly 304. Lock 334 secures the compression spring 314 axially. In a non-limiting example, as shown in FIG. 11, the lock 334 can include a shoulder bolt friction clamp 335 that passes orthogonally through a hole (not shown) in the piston 330. For example, cylinder 332 can include flat sides as an interface for the shoulder bolt 335 which are suitable to allow continuous locking of the spring 314. Although a shoulder bolt is shown as an example, other non-friction-based locking devices or friction-based locking devices can be relied upon to allow continuous locking of the spring.

The first and second unidirectional pulleys 326, 328 can include the first and second double drums 336, 338. The first and second double drums 336, 338 can be mounted in coincidence with the joint pin 310. Each of the first and second double drums 336, 338 are suitable to hold two cables 340, 342 each, such that each pulley 326, 328 has separate cables wrapped in opposing directions. For example, for the first linear shaft 306, a first cable 340a connects to an extension spring 344a that provides a torque on the pulley 326, while a second cable 342a is connected to an end 320 of the floating spring assembly 304. Similarly, for the second linear shaft 308, a first cable 340b connects to an extension spring 344b that provides a torque on the pulley 328, while a second cable 342b is connected to the other end 322 of the floating spring assembly 304.

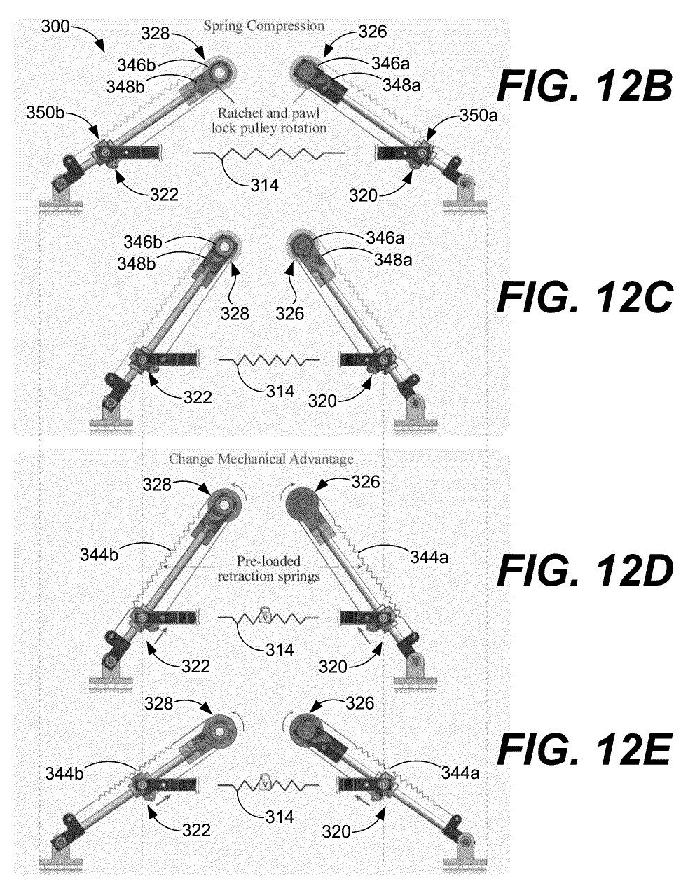

The leg structure 302 can also include a ratchet 346 and a pawl 348 at each of the first and second unidirectional pulleys 326, 328 suitable to lock rotation of each unidirectional pulley 326, 328 with respect to a pulley bracket (FIGS. 12A-12E). Each end of the floating spring assembly 304 can also include a linear ball bearing 350 (FIG. 11—bottom) in both ends 320, 322 that allow the floating spring assembly 304 to slide freely along the respective linear shafts 306, 308.

While the non-limiting example shown in FIG. 11 is one implementation, the structure can be relied on in other configurations and implementations. For example, the self-adjusting variable stiffness mechanism 300 can include means to secure the self-adjusting variable stiffness mechanism 300 to a leg of a user such that the hinge joint coincides with the knee of the user, one end of the first linear shaft 320 coincides with a hip of the user, one end 322 of the second linear shaft 308 coincides with the ankle of a user. In another example, a hand tool may include the self-adjusting variable stiffness mechanism 300. In yet another example, the self-adjusting variable stiffness mechanism 300 can be part of a transmission mechanism attached to a motor to drive heavy machinery by first accumulating energy with a small torque-limited motor and then releasing the accumulated energy to generate significant force beyond what would be possible using the same motor.

The method of energy accumulation using the self-adjusting variable stiffness mechanism 300 includes compressing the compression spring 314 housed in a floating spring assembly 304 to incrementally store energy. Energetic potential is normalized by a maximum energy that can be achieved in a single compression using the same maximal compression force. The method also includes repeatedly compressing the compression spring 314 and locking a length (l) of the floating spring assembly 304 between the end of one compression and the beginning of the next compression. The length (l) of the floating spring assembly 304 is defined by the distance between the two ends of the spring 320, 322. The energy stored by the compression spring 314 is retained between compressions. The floating spring assembly 304 is slidably attached to the first and second linear shafts 306, 308 and can lock to the compressed length between compressions. A force required to compress the compression spring 314 at the beginning of a repeated compression is lower than a constant gravitational force. In an example, a wearable exoskeleton can include the floating spring assembly and be suitable to be worn on a leg of a user. In the example of an exoskeleton, the compressing of the compression spring can include the user squatting.

### EXAMPLE 2

As described above, FIG. 11 depicts the spring-leg apparatus designed for the proposed energy accumulation task. The device includes three major sub-assemblies: the leg structure, the compression spring, and the spring retraction mechanism.

First, the leg structure 302 is formed by two linear shafts 306, 308 connected by brackets at the knee and pinned to create a hinge joint 312. The other ends 320, 322 of each shaft form the hip and ankle of the mechanism, as in FIG. 7.

Second, a compression spring 314 is housed in a piston-cylinder assembly (also referred to as the floating spring assembly herein) 304 where each end 316, 318 of the assembly connects to a linear ball bearing 350a, 350b that slides freely along the leg shafts 306, 308. In this example, to lock the spring axially, a shoulder bolt 335 passes orthogonally through a hole in the piston 330 and rides in a slot in cylinder 332, as shown in FIG. 11 (top). The cylinder features flat sides as an interface for the shoulder bolt 335, allowing continuous locking of the spring.

Finally, two unidirectional pulleys 326, 328, each comprising two drums 336, 338, are mounted in coincidence with the knee joint pin 310, as shown in FIG. 11 (bottom). One pulley is keyed to the knee pin, while the other pulley spins freely on the knee pin. Both, however, can spin freely with respect to the knee brackets. The double drums 336, 338 on each pulley 326, 328 feature separate cables 340, 342 wrapped in opposing directions. One cable 340a, 340b connects to an extension spring 344a, 344b that provides a torque on the pulley 326, 328, while the other cable 342a, 342b is connected to an end 320, 322 of the spring assembly 304. The system of pulleys serves to automatically shift the position of the spring assembly 304 while maintaining its orientation by balancing the forces of the two retraction springs 344a, 344b. Consequently, the mechanical advantage between the spring 314 and the leg is changed between each compression cycle.

FIGS. 12A-12E show the working principle of the apparatus of compressing the spring and changing the mechanical advantage of the leg over the spring 314 with the variable stiffness floating spring mechanism. FIG. 12A shows the CAD model of the apparatus 300 where the respective parts are labeled to help distinguish components responsible for the different endpoints of the spring assembly 304. Each leg segment utilizes a pulley 326, 328 and dual cable assembly 342, 344 to move the endpoints 350 of the spring assembly 304 in unison.

During compression, see FIGS. 12B and 12C, the spring 314 is unlocked and applies force on the bearing-mounted endpoints 350 of the spring assembly 304. Subsequently, the ratchet 346 and pawl 348 lock the rotation of each pulley 326, 328 with respect to their associated knee bracket. This cable-pulley setup then locks the position of the endpoints 320, 322 against the force of the spring. During compression, the endpoints 320, 322 of the spring are fixed along the leg segments 306, 308 by locking the rotation of each respective pulley 326, 328 via a ratchet 346 and pawl 348.

Following the spring compression, the spring 314 is locked, and the pre-loaded retraction springs 344a, 344b apply a torque on their respective pulleys 326, 328, see FIGS. 12D and 12E. To change the mechanical advantage, the mechanism extends back to its initial configuration with the spring 314 locked, allowing the pre-loaded retraction springs 344a, 344b to rotate each pulley 326, 328 and simultaneously retract the spring endpoints 320, 322 towards the hinge joint 312, which may coincide with a knee. Since the ratchet 346 and pawl 348 ensure the pulleys 326, 328 can only rotate in one direction, the torque on the pulley from the retraction spring 344 tends to pull the endpoints 320, 322 of the spring assembly 304 towards the knee joint 312. Therefore, as the mechanism returns to an initial configuration, the tension in the cables automatically shifts the endpoints of the spring toward the knee for a change in mechanical advantage before the next iteration.

Experimental Validation

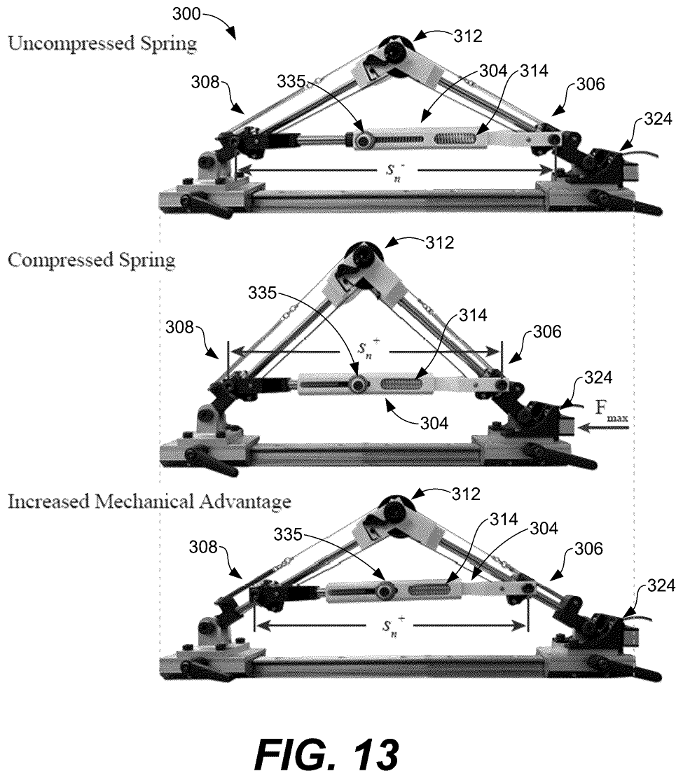

The apparatus 300, shown in FIG. 11, was mounted on a mechanical breadboard via a linear rail. The ends of each leg segment 306, 308 were connected to lockable carriages. One carriage was locked in place, acting as the ankle joint fixing the foot of the mechanism to the ground, while the other carriage could move freely along the rail like the hip joint. A load cell (MLP-50, Transducer Techniques) was mounted to a flat plate on the free-moving carriage to measure the force at the hip joint. FIG. 13 (top) shows the apparatus 300 in an uncompressed spring 314.

First, with the spring 314 unlocked, a force was applied manually to the load cell on the free slider until a pre-defined maximal force was reached, as shown in FIG. 13 (middle). At that point, the force was measured by the load cell. While manually applying force does not achieve constant static force during compression, the maximum applied force was held constant to represent the force boundary discussed above.

After collecting the force data, spring 314 was locked axially by tightening the friction clamp 335, and the length of the spring 314 was measured. Next, the slider was unlocked and moved back to its initial position, as shown in FIG. 13 (bottom). As described in FIGS. 12D and 12E, moving the slider shifted the spring assembly 304 to a new configuration that granted a greater mechanical advantage over the spring 314. The process described here was then repeated until maximum spring deformation was achieved.

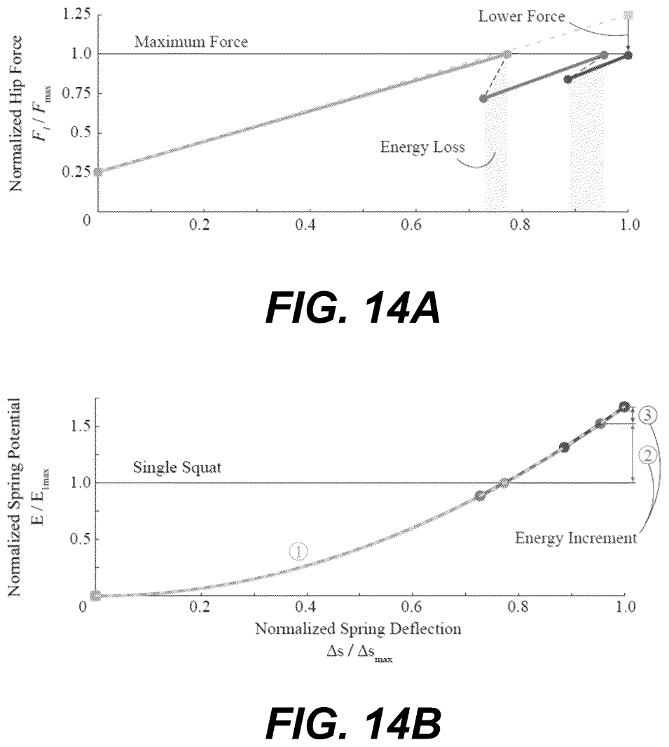

FIGS. 14A and 14B display the experimental result of the validation. FIG. 14A, shows the force-deflection trend predicted. Force is observed to increase up to the maximum force, then decrease to allow another squat despite the increased potential energy stored by the spring. The decrease in force required to enable a new squat is accomplished by the ratchet, pawl, and pulley assembly. The iterative force-deflection behavior (solid lines) is compared to the force required to reach the same spring deflection in a single squat (dashed line).

FIG. 14B shows the iterative increase in spring potential. The results show that the spring accumulates the same amount of energy in three squats (solid lines) as compared to that accumulated in one squat (dashed line). However, similar to what was predicted by the model, the mechanism reduces the maximal force necessary to accumulate the same amount of potential energy. In particular, the 25% decrease in maximal force observed in FIG. 14A resulted in 75% more energy accumulated by the spring compared to the energy one could store after a single squat when using the same maximal force. This result follows the same trend observed in FIGS. 10A and 10B. Further, when the device is reset to the initial mechanical advantage x=lₜ, it yields nearly 25% more assistive force (FIG. 14A) and 75% more energy compared to the maximum force used to repetitively compress the spring and the associated energy stored by the spring when compressed by the maximum force in a single squat (FIG. 14B).

While the experimental results were similar to the theoretical predictions, there were notable differences. For example, the mechanism exhibited roughly 84 percent efficiency, due to the energy loss observed during the experiment. This energy loss is shown in FIG. 14A by the black dashed lines not being vertical between iterations. The loss of energy can also be observed in FIG. 14B, where the energy accumulated in the spring first decreased at the beginning of each new energy accumulation cycle.

Two main factors contributed to the observed energy loss; first, the cables used to couple the retraction springs to the spring assembly were not completely inextensible, and second, the ratchet and pawl only provide discrete locking positions of the spring endpoints along the leg shafts and therefore introduced some amount of backlash.

One can also observe in FIG. 14A that force is not initially zero, see FIG. 14B. This initial force was due to the pre-loading of the compression spring to mitigate slack in the pulleys and cables. Also, the forces created by the retraction springs tend to pull the spring endpoints toward the knee, which in turn creates a moment about the knee that wants to straighten the leg.

In summary, a model of a lower-limb spring-leg exoskeleton is disclosed that may allow the human to perform a repetitive squat-to-stand task to accumulate energy. The squat-to-stand task was used as an example of iterative energy accumulation in a spring under force and deformation constraints. A variable stiffness floating spring leg mechanism is used to demonstrate the novel force-deflection and energy storage behavior that allows energy accumulation by repeated compression of the spring despite the same maximal force used in each compression cycle. Our theoretical predictions were experimentally validated using a variable stiffness floating spring mechanism.

The self-adjusting variable stiffness mechanism enables automatic adjustment of the mechanical advantage of the human or a robot over a spring between compression iterations. The mechanism demonstrated that a static gravitational force, provided by the mass of a human or robot, can be used to accumulate energy in a spring independent of the desired amount of energy stored by the spring. The self-adjusting variable stiffness mechanism and the associated energy accumulation method can be applied to implementations that include exoskeletons and spring-driven robots.

The new capability of iterative energy accumulation using a limited static force, could allow humans and robots with limited force capability and limited range of motion to perform physically demanding tasks, for example, to jump higher, move faster, or lift heavier objects, by harnessing the energy stored in assistive springs. The concepts can be relied upon for additional designs of robot exoskeletons and spring-driven robots with enhanced energy storage capabilities.

The above-described examples of the present disclosure are merely possible examples of implementations set forth for a clear understanding of the principles of the disclosure. Variations and modifications can be made without departing substantially from the spirit and principles of the disclosure. All such modifications and variations are intended to be included herein within the scope of this disclosure and protected by the following claims.

Therefore, the following is claimed:

1. A variable stiffness joint apparatus, comprising:
a torsional spring;
a variable stiffness mechanism comprising a self-locking mechanism and a linkage system, the self-locking mechanism comprising an auxiliary spring; and
an actuator in communication with the auxiliary spring of the self-locking mechanism, wherein when the actuator changes position, a force is applied to the auxiliary spring by the actuator and a stiffness is adjusted at an energy cost that is independent of the stiffness of the torsional spring and the energy stored by the torsional spring.

2. The variable stiffness joint apparatus of claim 1, wherein the energy is stored by the torsional spring without large or precisely timed forces provided by a motor or a human.

3. The variable stiffness joint apparatus of claim 1, wherein the torsional spring is attached to a joint, the linkage system is coupled to the torsional spring, the self-locking mechanism coupled to the linkage system.

4. The variable stiffness joint apparatus of claim 1, wherein the actuator controls the position of the self-locking mechanism, the position of the self-locking mechanism controls an arrangement of the linkage system, and the arrangement of the linkage system controls the stiffness of the joint apparatus.

5. The variable stiffness joint apparatus of claim 1, wherein the self-locking mechanism further comprises a linear ratchet, a pawl, a pawl support, and a pawl-release spring, the linear ratchet arranged orthogonal to and offset from a shaft, the pawl attached to the pawl support at a pivot point, the pawl in contact with the linear ratchet, the auxiliary spring coupled in series between the actuator and the pawl, the pawl-release spring attached to the pawl, the auxiliary and pawl-release springs offset from a pivot point of the pawl such that the auxiliary and pawl-release springs generate opposing moments on the pawl.

6. The variable stiffness joint apparatus of claim 5, wherein the pawl is controlled by the actuator through the auxiliary spring to make and break contact with the linear ratchet.

7. The variable stiffness joint apparatus of claim 5, wherein a level of stiffness of the joint based at least in part on a position of the pawl with respect to the linear ratchet.

8. The variable stiffness joint apparatus of claim 5, wherein when an applied force of the auxiliary spring is larger than a threshold force that keeps the pawl unlocked and a reaction force of the torsional spring, the pawl is moved to increase the stiffness of the joint.

9. The variable stiffness joint apparatus of claim 5, wherein when an applied force of the auxiliary spring is smaller than a threshold force and a reaction force of the torsional spring, the pawl is moved to decrease the stiffness of the joint.

10. The variable stiffness joint apparatus of claim 1, wherein the linkage system comprises a lever arm having a slot, a first linkage arm rotatable about a shaft and pivotably coupled to a first end of the lever arm, and a second linkage arm rotatable about the shaft and having a pin at a distal portion of the second linkage arm arranged to slide within the slot of the lever arm, the lever arm arranged such that a pivot point of the self-locking mechanism is positioned within the slot of the lever arm.

11. The variable stiffness joint apparatus of claim 1, further comprising a control device suitable for manual control of the actuator by a user.

12. The variable stiffness joint apparatus of claim 10, wherein the actuator of a control device comprises a Bowden cable and at least one lever to modulate the joint stiffness by moving the actuator.

13. The variable stiffness joint apparatus of claim 12, wherein the control device is handheld and operable by a finger or a hand of a user.

14. The variable stiffness joint apparatus of claim 1, wherein the torsional spring comprises a 3D printed carbon fiber reinforce torsional spring.

15. A method of changing a stiffness of a variable stiffness joint apparatus, comprising:
applying a force to an auxiliary spring of a self-locking mechanism, the self-locking mechanism coupled to the variable stiffness spring; and
changing a position of a pivot point of a self-locking mechanism based at least in part on the force applied to the auxiliary spring causing the stiffness of the variable stiffness spring to change.

16. The method of changing the stiffness of a variable stiffness joint apparatus of claim 15, wherein changing the position of the pivot point comprises moving a pawl on a linear ratchet of the self-locking mechanism.

17. The method of changing the stiffness of a variable stiffness joint apparatus of claim 16, wherein when the force of the auxiliary spring is larger than a threshold force to unlock the pawl and a reaction force of the torsional spring, the pawl is moved to increase the stiffness of the torsional spring.

18. The method of changing the stiffness of a variable stiffness joint apparatus of claim 16, wherein when the force of the auxiliary spring is smaller than a threshold force to unlock the pawl and a reaction force of the torsional spring, the pawl is moved to decrease the stiffness of the torsional spring.

19. The method of changing the stiffness of a variable stiffness joint apparatus of claim 15, wherein a large force and precise timing of the force applied by an actuator and the auxiliary spring is not required.

20. The method of changing the stiffness of a variable stiffness joint apparatus of claim 15, wherein applying a force to an auxiliary spring of a self-locking mechanism comprises changing a position of an actuator that acts on the auxiliary spring, the actuator being controlled by a user applying small and not precisely timed forces via a handheld control device.

21. A self-adjusting variable stiffness mechanism, comprising a compression spring, wherein energy stored by compressing the compression spring and the mechanism self-adjusts a stiffness to enable energy accumulation using a same maximal compression force which is not dependent on the energy accumulated in the compression spring.

22. The self-adjusting variable stiffness mechanism of claim 21, wherein repeated compressions increase an amount of energy stored.

23. The self-adjusting variable stiffness mechanism of claim 21, wherein the compression spring is housed in a floating spring assembly comprising a lock, wherein energy stored by the compression spring is retained by engaging the lock of the floating spring assembly to control endpoints of the compression spring.

24. The self-adjusting variable stiffness mechanism of claim 23, wherein the floating spring assembly further comprises a piston and a cylinder; and the floating spring assembly has two ends suitable to compress the compression spring.

25. The self-adjusting variable stiffness mechanism of claim 24, wherein the lock comprises a shoulder bolt that passes orthogonally through a hole in the piston, and the cylinder comprises flat sides as an interface for the shoulder bolt, or other non-friction-based or friction-based lock, suitable to allow continuous locking of the compression spring as the mechanism self-adjusts.

26. The self-adjusting variable stiffness mechanism of claim 22, further comprising:
a leg structure comprising first and second linear legs coupled to form a hinge joint; and
first and second unidirectional pulleys mounted in coincidence with the hinge joint;
wherein two ends of the floating spring assembly are slidably attached to the first and second linear legs, respectively; and the first and second unidirectional pulleys are configured to move the two ends of the floating spring assembly in unison.

27. The self-adjusting variable stiffness mechanism of claim 26, wherein a user extends the leg structure upon each compression cycle to reduce the stiffness of the leg structure before the next compression cycle.

28. The self-adjusting variable stiffness mechanism of claim 26, wherein the first and second unidirectional pulleys comprise first and second double drums mounted in coincidence with the hinge joint.

29. The self-adjusting variable stiffness mechanism of claim 28, wherein each of the first and second double drums are suitable to hold two cables each, such that each pulley has separate cables wrapped in opposing directions.

30. The self-adjusting variable stiffness mechanism of claim 28, wherein a first cable connects to an extension spring that provides a torque on the pulley, while a second cable is connected to an end of the floating spring assembly.

31. The self-adjusting variable stiffness mechanism of claim 26, wherein further comprising a ratchet and a pawl at each of the first and second unidirectional pulleys suitable to lock rotation of each unidirectional pulley with respect to a pulley bracket.

32. The self-adjusting variable stiffness mechanism of claim 21, wherein each end of the floating spring assembly further comprises a linear ball bearing that slides freely along the respective linear shafts.

33. The self-adjusting variable stiffness mechanism of claim 21, further comprising means to secure the self-adjusting variable stiffness mechanism to a leg of a user such that the hinge joint coincides with a knee of the user, an end first linear shaft coincides with a hip of the user, an end of second linear shaft coincides with the ankle of a user.

34. The self-adjusting variable stiffness mechanism of claim 21, wherein a hand tool comprises the self-adjusting variable stiffness mechanism.

35. A method of energy accumulation, comprising:
compressing a floating spring assembly comprising compression spring; and
storing energy in the compression spring locking the floating spring assembly to control endpoints of the compression spring.

36. The method of energy accumulation of claim 35, further comprising:
repeatedly compressing the compression spring; and
locking the floating spring assembly between the end of one compression and the beginning of the next compression, wherein the energy stored by the compression spring is retained between compressions.

37. The method of energy accumulation of claim 35, wherein a force required to compress the compression spring at the beginning of a repeated compression is lower than a constant force.

38. The method of energy accumulation of claim 35, wherein the floating spring assembly comprises end slidably attached to first and second linear legs, respectively.

39. The method of energy accumulation of claim 38, wherein a wearable exoskeleton comprises the floating spring assembly, the exoskeleton suitable to be worn on a leg of a user, and the compressing of the compression spring comprises the user squatting.

40. The method of energy accumulation of claim 35, wherein the self-adjusting variable stiffness mechanism is part of a transmission mechanism attached to a motor to drive heavy machinery, the method further comprising:
accumulating energy with a small torque limited motor; and
releasing the accumulated energy to generate a force.
